(19) United States
(12) Patent Application Publication
(10) Pub. No.: US 2010/0137967 A1
(43) Pub. Date: Jun. 3, 2010

(54) DEVICE FOR CONTROLLING A CATHETER

(75) Inventors: David Atlani, Jouy le Moutier (FR); Gonzague Issenmann, Clichy (FR)

Correspondence Address: OLIFF & BERRIDGE, PLC, P.O. BOX 320850, ALEXANDRIA, VA, 22320-4850, US

(73) Assignee: Stentys S.A.S, Paris (FR)

(21) Appl. No.: 12/452,046

(22) PCT Filed: Jun. 25, 2008
(86) PCT No.: PCT/IB2008/052546

§ 371 Date: Dec. 14, 2009

(30) Foreign Application Priority Data
Jun. 25, 2007 (FR) 0704541

(51) Int. Cl. A61F 2/84

(52) U.S. Cl. 623/1.11

(57) ABSTRACT

A device for deploying a stent, is connected to a catheter, the catheter being equipped with support structure for supporting the stent and with a deployment mechanism for deploying this stent. The device includes a portion connected to the stent-supporting structure and a portion connected to the stent-deploying mechanism. According to embodiments of the invention, the portion connected to the stent-supporting structure is in the form of a handle which may be grasped by a hand of the user, including at least one boss allowing it to be supported on a relatively stable surface, such as the operating table, the height of this boss being such that this support may be achieved in spite of the presence of the fingers of the user around the handle.

This application claims priority to French Patent Application No. 0704541, the entire contents of which are hereby incorporated herein by reference.

The present invention relates to a device for controlling a catheter used for deploying a stent.

It is known how to re-establish the diameter of a body lumen by placing in this lumen a radially expandable tubular frame currently called a “stent”, that may or not be self-expanding. For its implantation, a self-expandable stent is placed on an elongated support forming the axial core of a catheter and is maintained in a condition of radial contraction by a sheath that covers the stent, this sheath being slidably engaged on the support. The sheath may be slid relative to the support so as to release the stent.

The stent should be deployed at a specific location of the body lumen, particularly if it has to be implanted at a bifurcation or when it is used for implanting a heart valve.

In order to achieve deployment of the stent, the practitioner should implement a “fixed point”, i.e. maintain with one hand, a portion of the catheter fixed, connected to said support, and displace with his/her other hand another portion of the catheter connected to said sheath.

This standard, relatively empirical technique is not always very easy to apply and does not exclude a risk of inaccuracy as regards the positioning of the stent relatively to the body lumen upon deploying this stent.

Document US 2005/060016 describes a handle of a catheter for delivering a stent, which comprises a thumb wheel and a rack-and-pinion system in order to move back a restraining sheath of the stent, and a locking system providing immobilization of the rack. The thumb wheel is located so as to be found at the level of the thumb of the user when the handle is grasped. This handle may be grasped both by a right hand and a left hand.

The handle according to this prior document does not find a remedy to the aforementioned drawbacks.

An object of the present invention is to find a remedy to these drawbacks, by providing a control device that allows deployment of the stent in a specific location of the body lumen, and this in such a way that a user may easily apply it.

This device may be used for the deployment of a self-expanding stent. The device may comprise a catheter for conveying and deploying the stent, equipped with structure for supporting the stent and with a deployment mechanism for deploying the stent, the device further comprising a portion connected to said stent-supporting structure and a portion connected to said stent-deploying mechanism, said portion connected to the stent-supporting structure being in the form of a handle which may be grasped by a hand of the user.

According to embodiments of the invention, the handle comprises at least one boss allowing it to be supported on a relatively stable surface, such as the operating table, the height of this boss being such that this support may be achieved in spite of the presence of the fingers of the user around the handle.

With the handle according to embodiments of the invention, it is thereby possible to achieve the “fixed point” under the best conditions, while obtaining immobilization of this handle.

Advantageously, the handle comprises two front bosses, i.e. located at the end of this handle connected to the catheter, positioned transversely, i.e. substantially perpendicularly to the longitudinal axis of the handle.

Stability of the handle when it pivots according to this longitudinal axis is also obtained.

Preferably, the handle has a symmetrical shape relatively to a longitudinal median plane, so that it may be equally grasped by a right hand or a left hand of a user.

The handle may therefore be grasped in an undifferentiated way by a left or right hand depending on the side of the patient selected for approaching the implantation site.

Preferably,
the handle inwardly comprises a longitudinal guiding mechanism that comprises a rack, and a longitudinal aperture opening out into the area of the handle that is located opposite to the thumb of the user when the handle is grasped by the hand of this user;
the device comprises a portion connected to said stent-deploying mechanism, as a mobile slider along said guiding mechanism, this slider including a thumb wheel pivotably mounted thereon, connected to a pinion engaged with said rack, and an actuation button, this thumb wheel and this button protruding through said longitudinal aperture,
said rack being laid out in a location of the guiding mechanism such that said pinion is engaged with this rack on a first part of the actuation of said stent-deploying mechanism in the deployment direction of this stent, which substantially corresponds to a deployment of the stent, which still remains insufficient for immobilizing this stent relative to the body lumen, and such that said pinion is disengaged from this rack on a second part of the actuation of said stent-deploying mechanism in the deployment direction of this stent, which substantially corresponds to a deployment of this stent, sufficient for immobilizing this stent relative to the body lumen or at the very least limiting the mobility of this stent relative to this body lumen.

The device according to embodiments of the invention thus comprises a handle which may be firmly grasped by a hand of the user, the thumb of this hand will face said thumb wheel and said button once this grasping is performed; the thumb wheel may be actuated in rotation by the thumb of the user, in order to perform a slow and controlled backward movement of the slider on said first actuation part; this rotation therefore achieves a slow and controlled gradual deployment of the stent, while the handle properly remains in the hand of the user thanks to the actuation of this thumb wheel by the thumb, so as to ensure that the implantation location of the stent may be perfectly retained. Further rotation of the thumb wheel results in said pinion coming out of engagement with the rack; the button may then be used for performing faster actuation of said stent-deploying mechanism, and therefore fast deployment of the stent; this disengagement is however only performed once the stent has been sufficiently deployed in order to have a certain hold on the body lumen so as to be longitudinally immobilized relatively to this body lumen, or at the very least to have become slightly mobile relatively to the latter.

During these operations, the other hand of the user, freed by using the device according to embodiments of the invention, may be placed at the entry point of the catheter into the catheter guide or introducer and may allow, if necessary, fast correction of the positioning of the catheter.

The practitioner however keeps the option of acting either on the thumb wheel or directly on the actuation button, according to his/her preference; in this second case, by the fact that the thumb is engaged with the rack on said first part of the actuation of said deployment mechanism, a certain control of the deployment of the stent may be retained.

Preferably, the device may comprise an anti-rotation mechanism for preventing rotation of the thumb wheel in the opposite direction to the one allowing actuation of said stent-deploying mechanism in the direction of this deployment, or at the very least limiting free rotation of this thumb wheel in this opposite direction.

With the anti-rotation mechanism, it is possible to prevent or limit the elastic return of the stent-deploying mechanism in the direction opposite to the direction of deployment.

The anti-rotation mechanism may for example comprise friction against the thumb wheel and the slider and/or the rack, or a system of notches and pawl(s), or a system of bosses/cavities laid out on the adjacent faces of the thumb wheel and the slider and/or the rack, such that the passing of the bosses along each other during the rotation of the thumb wheel forms “hard points” which have to be crossed in order to allow this rotation.

Embodiments of the invention will be better understood, and other features and advantages of the latter will become apparent, with reference to the appended schematic drawings, illustrating as a non-limiting example, preferred embodiments of the relevant control device.

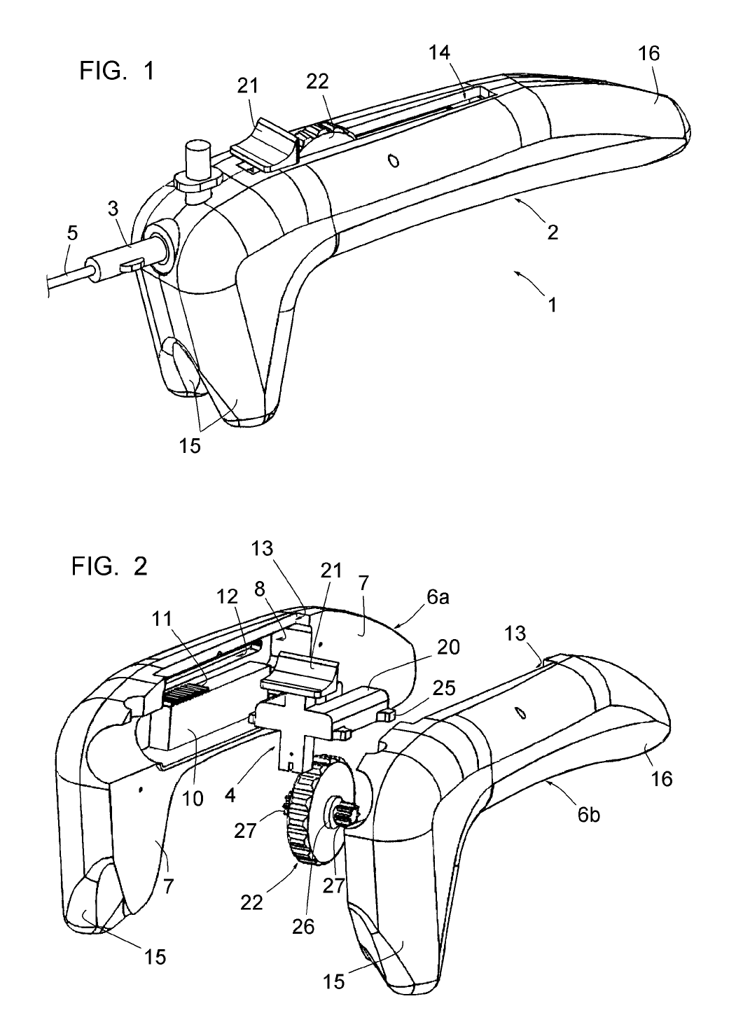

FIG. 1 is a perspective view of a device according to embodiments of the invention;

FIG. 2 is an exploded perspective view of the device of FIG. 1;

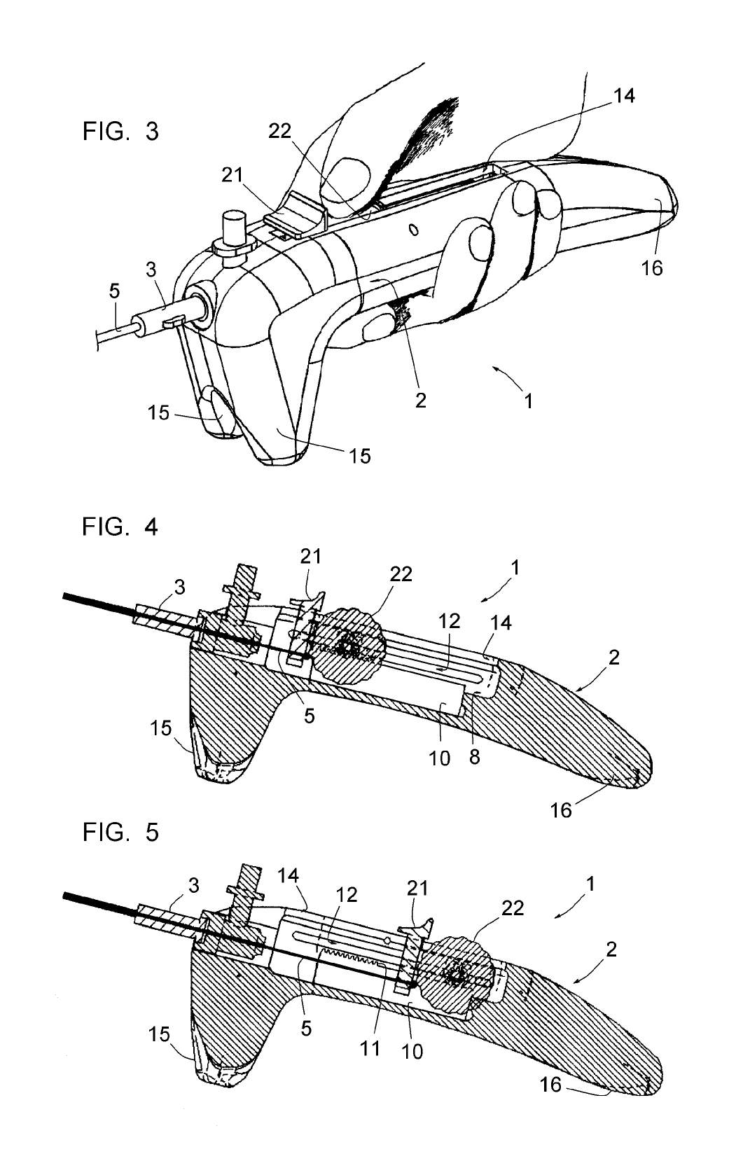

FIG. 3 is a view of it similar to FIG. 1 after the device is grasped by the hand of a user;

FIG. 4 is a median longitudinal sectional view of it, in a position of a device configured to include a slider, and

FIG. 5 is a view of a device, similar to FIG. 4, configured to include a slider.

The figures illustrate a device 1 for controlling a catheter used for deploying a stent.

The catheter (not shown) comprises an axial core forming a support for receiving the stent and a sliding sheath which, in a sliding position, covers the stent in order to keep it in a condition of radial contraction and which, in another sliding position, releases the deployment of this stent. The device 1 comprises a shell forming a handle 2, connected to said axial core through a protruding rod 3 which it comprises, and a slider 4 connected to a cable 5 itself connected to said sheath. The handle 2 may be grasped by a hand of a user, as shown in FIG. 3.

In the description hereafter, the terms of “proximal” and “distal” are taken into consideration relative to the point connecting the handle 2 to the catheter, “proximal” designating an area closer to this connection point and “distal” an area further away from this same point.

As this is more particularly apparent in FIG. 2, the handle 2 may be formed by two assembled half-shells 6a, 6b, which comprise planar surfaces 7 at their longitudinal end portions, so that they may come against each other in the position of assembly, and median recesses 8 for receiving the slider 4. These half-shells 6a, 6b, are preferably symmetrical relative to their assembly plane defined by the surfaces 7, this plane corresponding to the median longitudinal plane of the handle 2.

Each half-shell 6a, 6b comprises:
a block 10 laid out in the recess 8 on the front portion of the upper face of which a rack 11 is laid out;
a longitudinal groove 12 laid out sideways in the half-shell 6a, 6b along said upper face of the block 10; and
a longitudinal notch 13 laid out opposite this same upper face of the block 10, in the edge of the half-shell 6a, 6b; both notches 13 of both half-shells 6a, 6b thereby form a longitudinal aperture 14 which opens out into the area of the handle 2 located facing the thumb of the user when this handle 2 is grasped by the hand of this user.

Each half-shell 6a, 6b further forms a proximal boss 15 and comprises a slightly curved rear end 16. The height of each boss 15 is larger than the thickness of the fingers of a hand, as this is shown in FIG. 3, so that both transversely positioned bosses 15 which the handle 2 comprises after assembly, and half-shells 6a, 6b may be supported against a stable surface, for example, an operating table, without this support being an obstacle to the engagement of the fingers of the user other than the thumb around the handle 2.

The slider 4 preferably has a U-shaped body 20, including a median button 21 and receiving a thumb wheel 22.

The body 20 on its opposite longitudinal edges, includes protruding slides 25 that are able to be engaged and slideable in the grooves 12.

The median button 21 is connected to the body 20 by a portion able to be engaged and to slide in the aperture 14. It has a concave front shape adapted to the area supporting the thumb or index finger of the user.

The thumb wheel 22 is intended to be mounted in the space existing between both branches of the U which the body 20 forms. It comprises two axial pivots 26 intended to be received in housings which the body 20 comprises, forming bearings for receiving these pivots 26. The latter are axially extended by pinions 27 which will engage with both racks 11 when the slider 4 is placed between both half-shells 6a, 6b and when these half-shells are assembled together.

The slider 4 is thus mobile relative to the handle 2 between a front position shown in FIG. 4, corresponding to the position for covering the stent with the sheath, and a rear position shown in FIG. 5, corresponding to the position for having the stent completely released by the sheath.

As apparent in FIGS. 2, 4 and 5, both racks 11 may only occupy a front portion of the length of the recesses 8. Their lengths may be such that the pinions 27 are engaged with them on a first portion of the sliding course of the sheath, substantially corresponding to a deployment of the stent, remaining still insufficient for immobilizing this stent relatively to the body lumen into which the catheter is engaged, and that the pinions 27 are disengaged from them on a second portion of the sliding course of the sheath, substantially corresponding to a deployment of the stent, sufficient for immobilizing this stent relatively to the body lumen.

In practice, the catheter is introduced into the body lumen until its stent-supporting portion is positioned at the implantation site. The handle 2 is then grasped by a hand of the user and placed on a stable surface, for example, the operating table. This grasping is preferably accomplished in the way shown in FIG. 3, in which four fingers of the hand may be engaged around the handle 2 and the thumb arriving at the level of the thumb wheel 22 and the button 21. The other hand of the user may be placed at the entry point of the catheter into the catheter guide and may if necessary allow fast correction of the positioning of the catheter.

The thumb wheel 22 is then actuated into rotation by the thumb of the user, in order to perform a slow and controlled backward movement of the slider 4 on said first portion of the sliding course. This rotation therefore achieves a slow and controlled gradual deployment of the stent, while the handle 2 remains properly held in the hand, which ensures that the implantation location of the stent may be retained. Further rotation of the thumb wheel 22 results in the pinions 27 disengaging from the racks 11; the button 21 may then be used for performing faster sliding of the sheath, and therefore faster deployment of the stent. This disengagement however may be performed only once that the stent has been sufficiently deployed in order to have a certain hold on the body lumen so as to be longitudinally immobilized relatively to this body lumen, or at the very least to have become slightly mobile relatively to the latter.

The practitioner keeps the option of acting either on the thumb wheel 22 or directly on the button 21 to perform a backward movement of the sheath; in this second case, by the fact that the thumb wheel 22 is engaged with the racks 11 on said first part of the sliding course, it is possible to retain a certain control on the deployment of the stent.

Embodiments of the invention thus provide a device for controlling a catheter used for deploying a stent having, as compared with the homologous devices of the prior art, the decisive advantage of being perfectly adapted for allowing deployment of the stent in a specific location of the body lumen, and this in such a way that a user may easily apply it.

The invention was described above with reference to embodiments given as examples. It is obvious that it is not limited to these embodiments but it extends to all the other embodiments within the scope and spirit of the present disclosure.

1. Device for controlling a catheter used for deploying a stent, the catheter being equipped with structure for supporting the stent and with a deployment mechanism for deploying this stent, the device comprising a portion connectable to said stent-supporting structure and a portion connectable to said deployment mechanism, said portion connectable to the stent supporting structure being in the form of a handle which may be grasped by a hand of the user, wherein the handle comprises at least one boss allowing it to be supported on a relatively stable surface, such as an operating table, the height of this boss being such that this support may be achieved in spite of the presence of fingers of the user around the handle.

2. Device according to claim 1, wherein the handle comprises two front bosses located at an end of this handle connected to the catheter, positioned substantially perpendicularly to a longitudinal axis of the handle.

3. Device according to claim 1, wherein the handle has a symmetrical shape relatively to a longitudinal median plane, so that it may be equally grasped by a right hand or a left hand of a user.

4. Device according to claim 1, wherein:
the handle inwardly comprises a longitudinal guiding mechanism that comprises a rack, and a longitudinal aperture opening out into an area of the handle located opposite to a thumb of the user when the handle is grasped by the hand of the user; and wherein
said portion connected to said stent-deploying mechanism comprises a mobile slider along said guiding mechanism, which includes a thumb wheel pivotably mounted thereon, connected to a pinion engaged with said rack, and an actuation button protruding through said longitudinal aperture,
said rack being laid out in a location of the guiding mechanism such that said pinion is engaged with the rack on a first part of the actuation of said stent-deploying mechanism in a deployment direction of the stent, which substantially corresponds to a deployment of the stent, which still remains insufficient for immobilizing this the stent relative to a body lumen, and such that said pinion is disengaged from the rack on a second part of the actuation of said stent-deploying mechanism in the deployment direction of the stent, which substantially corresponds to a deployment of the stent, sufficient for immobilizing the stent relative to the body lumen o limiting the mobility of the stent relative to this body lumen.

5. Device according to claim 1, wherein the handle is formed by two assembled half-shells.

6. Device according to claim 5, wherein the two half-shells are symmetrical relatively to an assembly plane, this plane corresponding to the median longitudinal plane of the handle.

7. Device according to claim 5, wherein each half-shell comprises:
a block laid out in a median recess on a front portion of an upper face of which a rack is laid out, and
the thumb wheel comprises two pinions engaging with both racks of both half-shells when these half-shells are assembled together.

8. Device according to claim 5, wherein each half-shell comprises a longitudinal groove, both grooves of both half-shells forming said longitudinal guiding mechanism.

9. Device according to claim 5, wherein each half-shell comprises a longitudinal notch both notches of both half-shells forming said longitudinal aperture.

10. Device claim 1, comprising an anti-rotation mechanism for preventing rotation of the thumb wheel in the opposite direction to the direction allowing actuation of said stent deploying mechanism in the direction of this deployment, or limiting free rotation of this thumb wheel in this opposite direction.

11. A method for controlling deployment of a stent, the method comprising:
grasping a handle of a device and supporting the handle on a stable surface;
and retracting a slider of the device.

12. The method according to claim 11, wherein the retraction of the slider is activated by rotating a wheel of the device.

13. The method according to claim 11, wherein the retraction of the slider is activated by pressing a button of the device.

14. The method according to claim 11, wherein the retraction of the slider is activated by at least one of rotating a wheel of the device and pressing a button of the device.

15. The method according to claim 12, wherein rotation of the wheel causes a pinion to be disengaged from a rack.

16. The method according to claim 12, further comprising preventing the rotation of the wheel in a direction that is opposite the direction of the rotation of the wheel for activating the retraction of the slider.
